(19) United States
(12) Patent Application Publication
(10) Pub. No.: US 2025/0135066 A1
(43) Pub. Date: May 1, 2025

(54) METHOD AND SYSTEM FOR ASSOCIATING SECONDARY MATERIALS WITH A TISSUE GRAFT AND MODIFIED TISSUE GRAFTS

(71) Applicant: Barry Markman, Frisco, TX (US)

(72) Inventor: Barry Markman, Frisco, TX (US)

(21) Appl. No.: 18/585,269

(22) Filed: Feb. 23, 2024

Related U.S. Application Data
(60) Provisional application No. 63/593,177, filed on Oct. 25, 2023.
(60) Provisional application No. 63/597,243, filed on Nov. 8, 2023.

(51) Int. Cl. A61L 27/36; A61L 27/54

(52) U.S. Cl. CPC A61L 27/3633; A61L 27/54; A61L 2400/06

(57) ABSTRACT

Devices and systems are provided for associating secondary materials with tissue grafts, both ex-situ and in-situ. The secondary materials may be medicants, an external cellular matrix factor, a cell, an enzyme, or an element or compound, such as oxygen. In a method, different secondary materials are delivered to a tissue graft via a delivery device at different times, such as at different stages of a healing process. The delivery device may be associated with a wound cover or bandage.

### RELATED APPLICATION DATA

This application claims priority to U.S. Provisional Application Ser. No. 63/593,177, filed Oct. 25, 2023, and U.S. Provisional Application Ser. No. 63/597,243, filed Nov. 8, 2023, which applications are incorporated herein by reference in their entirety.

### FIELD OF THE INVENTION

The present invention relates to tissue grafts.

### BACKGROUND OF THE INVENTION

Wound dressings and tissue grafts are commonly used to treat areas of injury, such as the skin. However, existing wound dressings and tissue grafts, and the use thereof, suffer from a number of drawbacks. For example, in the current state of the art, wound dressings and biologic tissue grafts are static in nature. In general, they address a specific phase of healing in that their content is not exchangeable.

Healing of a wound or a transplanted organ must undergo several phases to completion. In wound healing, The first two critical phases are the inflammatory and proliferative phases. Each phase requires different medicants toward a successful end result.

For example, during the initial inflammatory phase of wound healing, medicants directed toward cellular elements to remove compromised tissue are required. Other medicants that assist stem cells in the formation of new microvasculature between the tissue graft and host site is beneficial during the inflammatory phase. Lastly, an oxygen rich environment is beneficial to promote faster healing, healing, better incorporation, greater exchange between the modified tissue graft and the host.

In contrast, during the later proliferation phase of wound healing, the recruitment of fibroblasts to enhance a structural matrix and formation of micro-vessels (Angiogenesis), is critical.

A second example pertains to a transplanted organ. Rejection of the transplanted organ occurs in the early stages after surgery. Introduction of medicants relevant to prevent immunological rejection by the host are critical to this early phase. Secondarily, the physiologic functioning of the transplant organ occurs is the next phase.

Current tissue dressings (both synthetic and biologic), can also be categorized as being singular or composite in structure. Singular tissue grafts may be limited in that they contain a primary element, such as a gel (such as a hydrogel or colloids). A composite graft can contain a gel as well as, a fibrous matrix, stems cells, and a antibacterial agent (an Ag/Antibiotic).

Current dermal tissue grafts are categorized as being acellular or decellular. Current placental grafts are categorized by the elements with the graft, i.e. amnion, chorion, umbilical chord, etc.

In each case, the dressing or graft is static in structure, and may not provide critical medicants, external cellular matrix factors, cells, enzymes, and/or other secondary elements or materials, such as oxygen, which have advantages at different phases of the healing process.

Current surgical transference and incorporation of medicants or other materials critical for wound healing also rely on a normal oxygen environment in the host tissue, which often does not exist. A normal oxygen environment in the wound is dependent on multiple patient variables factors. One of the critical variables is arterial inflow and venous outflow in the host wound site. A hypoxic wound environment my limit the exchange, incorporation of elements that assist in healing of the wound.

However, the matrix within the dermal layer in the skin or tissue graft is complex, relatively dense in structure, and tissue grafts contain multiple anatomic structures. Examples of these dermal structures within the matrix, include but not limited to hair follicle germinal centers, pigment stem cells, sebaceous glands, sweat glands, nerve corpuscles micro sensory endings, and cells supporting the regeneration of the matrix.

Thus, various problems exist when attempting to solve the problems associated with existing wound dressings and tissue grafts.

### BRIEF DESCRIPTION OF THE FIGURES

FIGS. 1A-1C illustrate a method of associating secondary materials with a tissue graft in accordance with an embodiment of the invention;

FIGS. 2A and 2B illustrate embodiments of a device for use in associating secondary materials with a tissue graft in accordance with embodiments of the invention;

FIGS. 3A and 3B illustrate other embodiments of a device for use in associating secondary materials with a tissue graft in accordance with the invention;

FIG. 4 illustrates a system for associating secondary materials with a tissue graft in accordance with an embodiment of the invention;

FIG. 5 is a bottom view of a device for associating secondary materials with a tissue graft of the system illustrated in FIG. 4; and

FIG. 6 illustrates another embodiment of a device for associating secondary materials with a tissue graft in accordance with the invention.

### SUMMARY OF THE INVENTION

One aspect of the invention is a tissue graft having associated secondary materials, such as, but not limited to one or more of: a medicant, an external cellular matrix factor, a cell, an enzyme, or element or compound (such as oxygen). Another aspect of the invention comprises a device or system for associating one or more secondary materials with a tissue graft, such as a delivery mechanism.

The delivery mechanism may comprise an apparatus including a plurality of delivery members, such as injectors, delivery needles, or the like, and one or more sources of secondary materials. In one embodiment, the delivery mechanism may be part of a delivery system which includes a wound bandage, cover or other treatment elements.

In some embodiments, the secondary materials may be associated with a modified tissue graft where at least one surface of the graft has been modified.

Another embodiment of the invention comprises methods of associating secondary materials with a tissue graft. In one embodiment of a method, different secondary materials are associated with a tissue graft at different times, such as during different periods of a healing process.

Further objects, features, and advantages of the present invention over the prior art will become apparent from the detailed description of the drawings which follows, when considered with the attached figures.

### DETAILED DESCRIPTION OF THE INVENTION

Some aspects of the invention comprise devices and systems for associating secondary materials with tissue grafts, both ex-situ and in-situ. Additional aspects of the invention comprise modified tissue grafts, including those which have secondary materials associated therewith. Yet other aspects of the invention comprise methods of associating secondary materials with tissue grafts and methods of creating modified tissue grafts, including methods of treatment.

The present invention has applicability to tissue grafts. The tissue grafts may comprise cadaveric human tissue, harvested human tissue, or may comprise amniotic or chorionic tissue (including genetically altered human de-cellular amniotic and/or chorionic tissue), or other tissue. In other embodiments, the tissue grafts may comprise cellular non-human tissue, including cellular and acellular processed grafts. The tissue grafts may also comprise synthetic materials. In some embodiments, the tissue grafts may also comprise one or more layers.

In one embodiment, one or more secondary materials may be associated with a tissue graft. The one or more secondary materials may comprise one or more of a gas, liquid, or other similar material (gel) or may comprise materials which are associated with a gas, liquid or gel, including encapsulators, acting as a carrier. The one or more secondary materials may comprise, but are not limited to, one or more medicants, external cellular matrix factors, cells, enzymes, oxygen or other elements, compounds or materials. As described below, the secondary materials may be selected for association with the tissue graft for particular purposes and at particular times, such as relating to different phases of a healing process, different conditions, different anatomical areas and different associated host tissue functionality.

In one embodiment of the invention, one or more secondary materials may be associated with a modified tissue graft. Such a graft may be modified in various manners, such as disclosed in U.S. Patent Application Publication 2021/019290 to the inventor herein, which prior application is incorporated by reference in its entirety herein. As disclosed therein, a tissue graft may be modified to include one or more surface features, such as to create one or more voids, openings or the like in one or more surfaces of the tissue graft.

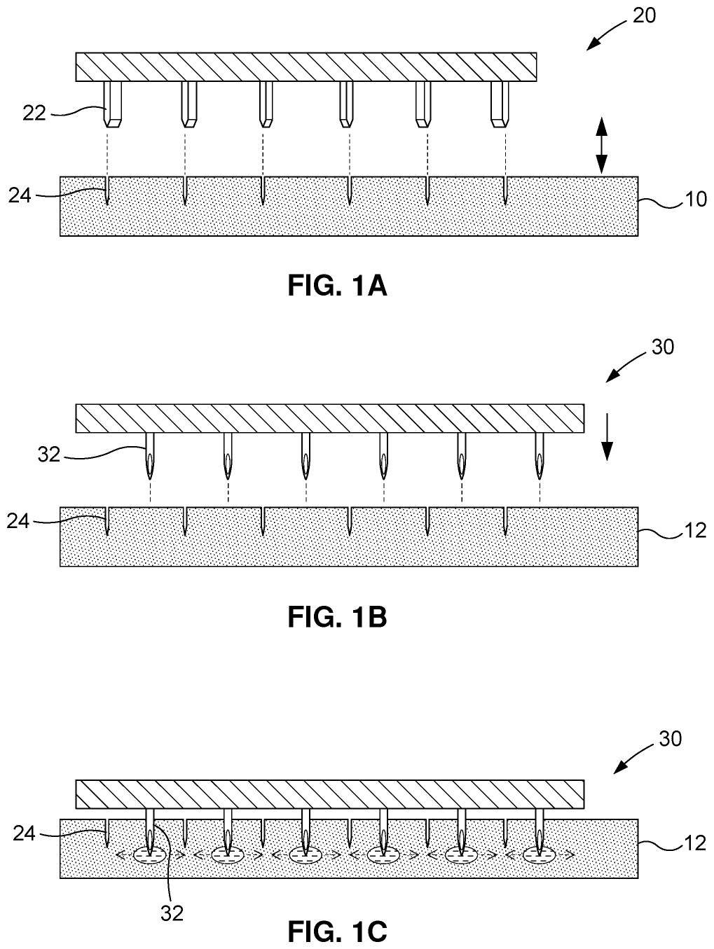

For example, as illustrated in FIG. 1A, a cutting die 20 having a plurality of downwardly extending blades 22 may be pressed into engagement with a tissue graft 10, to create a plurality of cuts 24 (or other openings/voids, etc.), thereby creating a modified tissue graft.

One or more secondary materials might then be associated with the modified tissue graft 12. In one embodiment, secondary materials may be delivered to a tissue graft utilizing a delivery system or delivery device.

For example, as illustrated in FIG. 1B, a delivery device 30 may comprises one or more delivery members, such as inserters, injectors, blades, cutters or combinations thereof, which may be referred to herein as an integrated blade/needle design 32 or simply as a “needle”, which are used to inject or otherwise associate the one or more secondary materials with (and preferably, within—e.g., into an interior thereof below the exterior surfaces of) the modified tissue graft 12. It will be appreciated that in the preferred embodiment in which the secondary materials as associated with an interior portion of a tissue graft, the integrated blade/needle design 32 is preferably configured to pierce, cut or otherwise penetrate the tissue graft. In some embodiments, the integrated blade/needle design 32 may be in the form of blades which cut the tissue graft and also deliver secondary materials, such as via common elements, or in other embodiments, where blades or other cutters are used to cut the tissue graft and other elements, such as needles are used to associate the secondary materials with the tissue graft. Additional details of an example delivery device and system are provided below.

In one embodiment, the delivery device 30 may be used to deliver the secondary materials to regions of the modified tissue graft 12 which have not been modified. For example, relative to a modified tissue graft 12 having a plurality of cuts, the delivery device 30 may be used to deliver the secondary materials to areas of the modified tissue graft 12 that have not been cut (such as between the cuts). This configuration aids in ensuring that the secondary materials do not simply exit the modified tissue graft 12 through the modified area thereof. Instead, as illustrated, the secondary materials may diffuse through the modified tissue graft 12, such as drawn away from the area of injection/introduction and towards the modified areas. This aids in dispersion of the secondary material through the tissue graft. In the illustrated configuration, a first apparatus is used to cut or modify the tissue graft 10 and then a second device is used to deliver the secondary materials to the modified tissue graft 12. In another configuration, however, a single device might include the integrated blade/needle design 32, such as having both downwardly extending blades and delivery needles which are spaced from the blades.

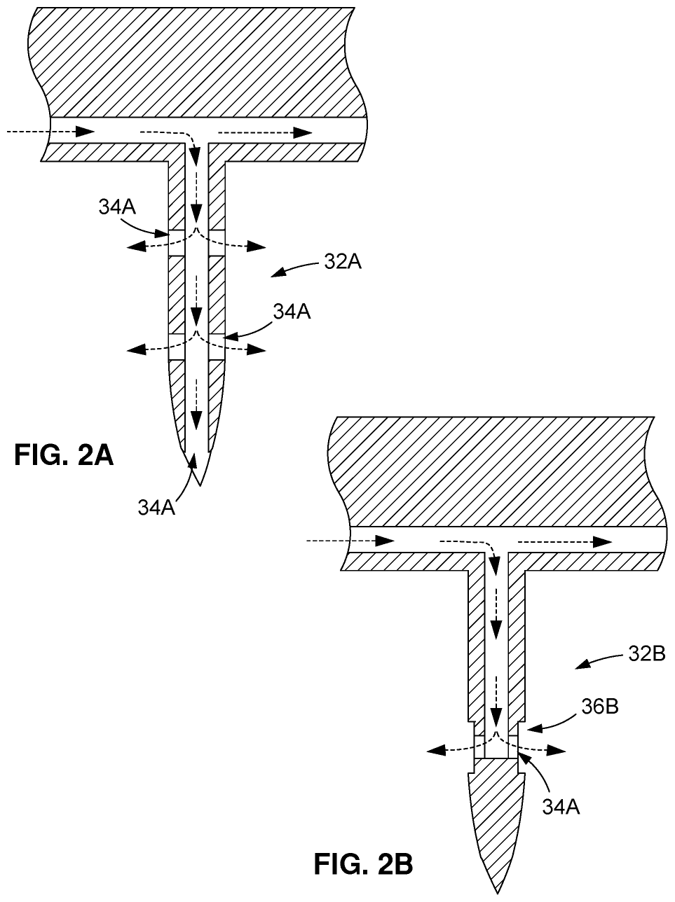

In another embodiment, as illustrated in FIG. 2A, dispersion of the secondary material may be effectuated by delivery through an integrated blade/needle design 32A having a plurality of delivery ports 34A, such as in one or more locations in the sidewall thereof, including where the size and/or shape of the ports 134 may vary. It will be appreciated that this embodiment integrated blade/needle design 32A might be used to associated secondary materials with a tissue graft 10 or a modified tissue graft 12. In some embodiments, a tip or distal end of the integrated blade/needle design 32A might be configured to penetrate the tissue graft 10, while in others, the tip might be configured as a blade or other implement, so as to cut or otherwise modify the tissue graft.

In yet another embodiment, as illustrated in FIG. 2B, a integrated blade/needle design 32B may include one or more features for creating a void within the tissue graft. For example, the integrated blade/needle design 32B may define an inset 36B, such as in the sidewall thereof, where one or more delivery ports 34B are located. When the integrated blade/needle design 32B is inserted into the tissue graft, a void is created due to the inset, creating a space for the infusion or injection of the secondary materials. It will be appreciated that this embodiment integrated blade/needle design 32B might be used to associated secondary materials with a tissue graft 10 or a modified tissue graft 12.

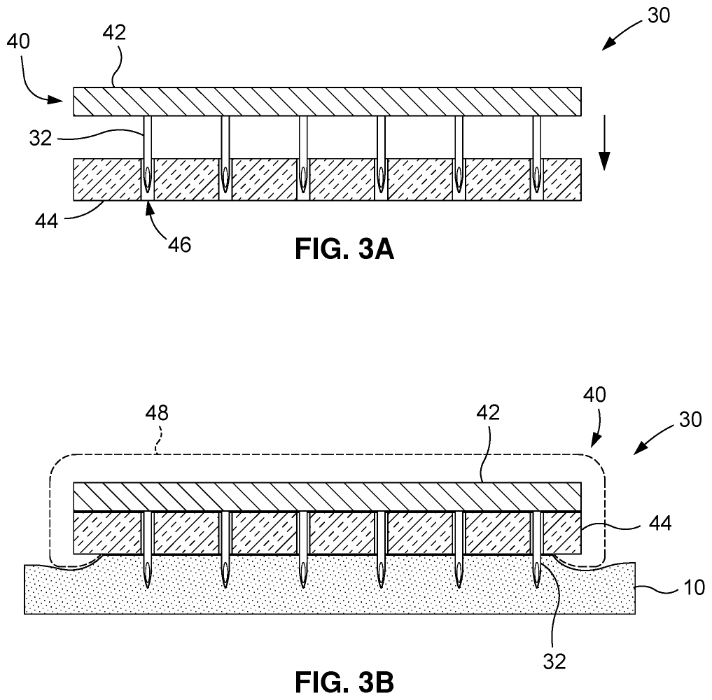

In one embodiment, as illustrated in FIG. 3A, the delivery device 30 may comprise an apparatus or jig 40, such as a mount, to which the integrated blade/needle design 32 are mounted and extend. In one embodiment, the integrated blade/needle design 32 may be retractable, such as to prevent accidental integrated blade/needle design sticks. In one configuration, the integrated blade/needle design 32 might be retractable into and out of the jig 40. For example as illustrated in FIG. 3A, the jig 40 might comprise a base 42 to which the integrated blade/needle design 32 are mounted, and a guide 44 which defines a plurality of slots or passages 46 through which the integrated blade/needle design 32 may be extended (such as from a top side of the guide 44 through to an opposing bottom side thereof), where the base 42 is movable relative to the guide 44. The base 42 may be removable from a first or retracted position in which a distal end of each integrated blade/needle design 32 is located in the slots 46, to a second or extended position in which the distal end of each integrated blade/needle design 32 extends outwardly of the guide 44, such as for insertion into a tissue graft.

In some embodiments, the base 42 might be biased away from the guide 44 towards the retracted position. This then requires the user to actively press the base 42 towards the guide 44 in order to move the integrated blade/needle design to their extended position. In some embodiments, a retaining mechanism or lock might be used to maintain the integrated blade/needle design in their extended position, such as when the delivery mechanism 30 is in use.

The base 42 (or the entire jig 40) might be manually moved or it might be moved by a mechanism, such as a motor driven actuator. Such an actuator might include a movement control, such as which controls the range of movement so as to control the depth of insertion of the integrated blade/needle design 32. In another embodiment, the jig 40 (or the base 42 relative to the guide 44) might include one or more stops, such as to control the depth of insertion of the integrated blade/needle design 32. The stops might be position adjustable, such as to allow the depth of insertion to be varied. For example, different guides 44 might be associated the a particular base 42, where the guides 44 have different thicknesses or depths, thus controlling the distance which the integrated blade/needle design 32 extend therefrom.

One aspect of the invention is an off-loading mechanism for a secondary material delivery device. Increased pressure and the effect of shear forces of boney prominences beneath skin and soft tissue, is one of the main reasons for the development of pressure injuries. The most common bone prominences that cause pressure injuries include, but not limited to, the sacrum, hip, and ischial bones. The delivery device 30 may include or incorporate an off-loading mechanism, such as to distribute mass/pressure away from the tissue graft 10.

In some embodiments, the off-loading mechanism may comprise an extension of the delivery device 30, such as at the periphery thereof (such as a peripheral extension of the guide 44), which periphery is located around an interior area which is aligned with the tissue graft 10. In such a configuration, mass/pressure applied to the delivery device 30 may be routed through the peripheral areas to and from tissue surrounding the tissue graft 10, rather than the graft itself.

In another configuration, the off-loading mechanism might comprise or include one or more pads or cushions which are associated with the delivery device 30. For example, as illustrated in FIG. 3B, a cushion 48 might be associated with the delivery device 30, such as the base 42 and/or guide 44. The cushion 48 may comprise a compressible material, such as foam or the like. In other embodiments, the cushion 48 might extend over the sides of the delivery mechanism 30 and/or a portion of the bottom, such as where the delivery mechanism fits into a recess formed in the cushion 48. This embodiment may be beneficial in situations where the delivery mechanism 30 is used in-situ.

As indicated above, one aspect of the invention is a delivery system for delivering secondary materials to a tissue graft, and preferably into a tissue graft, such as via one or more delivery members such as an integrated blade/needle design. In one embodiment, the delivery system includes the delivery device and a source of secondary materials, plus a means for delivering the secondary materials to the delivery device.

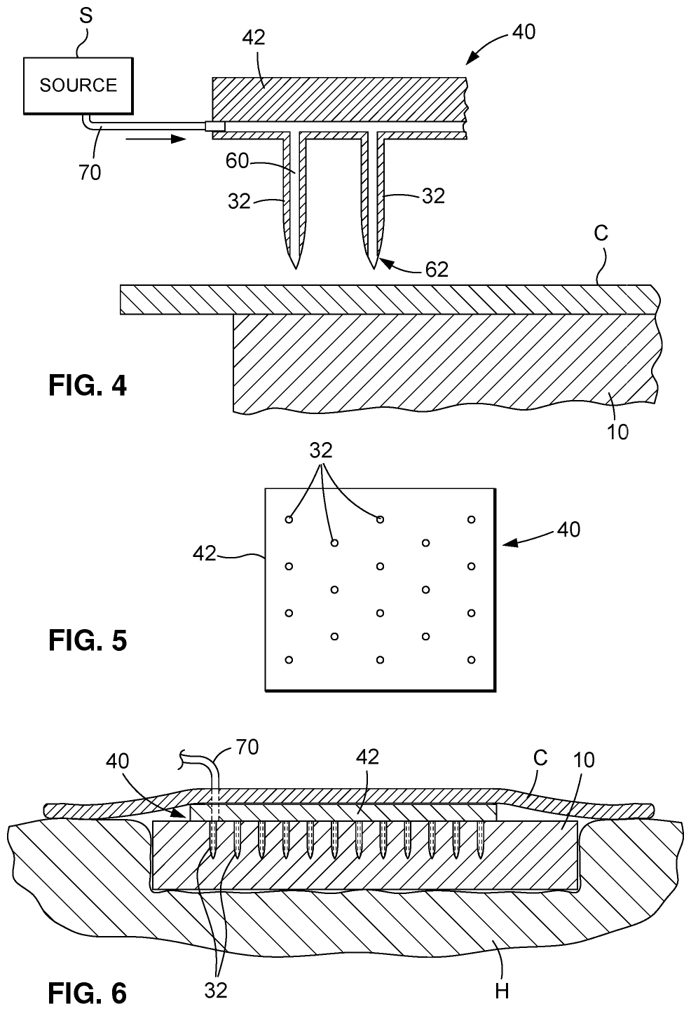

Referring to FIG. 4, in one embodiment, the integrated blade/needle design 32 includes at least one delivery passage 60 which extends from at least one secondary material source to the at least one delivery port 62 (as described above, such a port may be located in a sidewall of the integrated blade/needle design 32). In one embodiment, the passage 60 may extend from the port 62, such as a through the integrated blade/needle design 32, whereby the integrated blade/needle design effectively defines an elongated axially extending central passage surrounded by a sidewall.

As indicated above, the integrated blade/needle design 32 may be associated with a jig 40 which supports a plurality of the integrated blade/needle design 32, such as illustrated in FIG. 5. The number of delivery integrated blade/needle design 32, including their arrangement, may vary, such as depending upon the application. In one embodiment, as illustrated in FIG. 5, the proximal end of each integrated blade/needle design 32 may be connected to the base 42 of the jig 40, such as where the integrated blade/needle design 32 extend outwardly from a bottom side thereof.

In one embodiment, the delivery passage 60 connects to at least one secondary material source S. For example, the source S might be a tank or container of secondary material. At least one inlet or line 70 may lead from the source S to each delivery passage 60, such as through the base 42. In some embodiments, the delivery passages 60 of more than one integrated blade/needle design 32 might be connected to a single delivery line, or independent lines might be provided from one or more sources to each integrated blade/needle design 32 and associated passage 60. In some embodiments, the lines 70 may be routed (including having shapes) to promote laminar flow of the secondary materials therethrough.

In one embodiment, as illustrated in FIG. 4, one or more secondary materials may be associated with a tissue graft 10 via the integrated blade/needle design 32. In particular, the jig 40 and tissue graft 10 are preferably moved relative to one another, such as by lowering the jig 40 downwardly until the distal ends of the integrated blade/needle design 32 are located within the tissue graft 10. Secondary materials may then be delivered from the source S to the tissue graft 10 via the one or delivery ports 62 of the integrated blade/needle design 32.

In one embodiment, the source S of secondary materials may be pressurized (such as a pressurized tank or container) or a pump may be used to deliver the secondary material from the source S to the tissue graft 10. In one embodiment, the source S might comprise a hospital source, such as an oxygen delivery system in a hospital, or might comprise individual sources, such as containers or tanks. In some embodiments, a single source may be used to deliver secondary materials to a plurality of the integrated blade/needle design 32. In other embodiments, a source may be provided relative to each (or several) integrated blade/needle design 32. For example, in a portable configuration, the delivery device 30 might enable connection of a source to the jig 40, such as by connecting a cannister of pressurized oxygen to a port defined by or connected to the base 42, which port leads to (such as via one or more lines or passages) the integrated blade/needle design 32.

As indicated above, aspects of the invention may be utilized to modify a tissue graft ex-situ. For example, a tissue graft may be obtained and have secondary materials associated with it before it is placed. As one example, the tissue graft may be obtained, have the secondary materials associated therewith, and then be packaged at one location and then be delivered to another location (such as a hospital) for use, such as application to a patient with a wound.

In another example, as illustrated in FIG. 6, a tissue graft 10 having secondary materials associated therewith might be located under or be associated with another body, such as a secondary tissue layer or a cover C. Such as cover C might comprise, for example, a bandage. The combination bandage with tissue graft may be delivered for use as a package.

In other embodiments, however, the systems and device of the invention may also be used to deliver secondary materials to a tissue graft in-situ. In one configuration, the delivery device 30 might be associated with or include a wound covering or wound treatment package (such as which may include various other features or elements). In such a configuration, for example, the jig 40 might be located under a cover C thereof. When placed, as illustrated, the integrated blade/needle design 32 may extend into the tissue graft 10 as associated with a host H (such as a patient), under a cover C thereof. Secondary material may then be delivered to and associated with the tissue graft 10, including continuously or at one or more times. In some embodiments, a vacuum or suction may be applied, such as to induce circulation of material. In other embodiments, a pressurized environment might be created under the cover C. In such a configuration, the delivery device might be configured as an integrated bandage, wound treatment device or the like which is manufactured and sold as unit and is then associated with the tissue graft once the graft is associated with the host.

Of course, the secondary material delivery device 30 or system might have other configurations and variations. For example, the integrated blade/needle design 32 or other delivery elements might be of varied sizes, including lengths (such as to deliver secondary materials at different depths into the tissue graft). The delivery device 30 might be used to deliver different secondary materials, such as the same time (such as from two sources at the same time) or at different times. Further, while the delivery mechanism has been illustrated as being used relative to one side of a tissue graft, the delivery mechanism might be configured to associated secondary materials with a tissue graft from both sides, such as where the jig has a top portion and a bottom portion with associated delivery integrated blade/needle design which are inserted into a top and bottom of the tissue graft at the same or different times.

In particular, as one aspect of the invention, the delivery device 30 may be used to deliver different secondary materials to a tissue graft 10 at different times, such as different secondary materials during different wound healing phases.

In one embodiment of the invention, the secondary materials may comprise oxygen. In one embodiment, this may be done to increase the minimum oxygen tension the tissue graft to >40 mm/hg or >21% O₂ concentration, but not to the point of oxygen toxicity to host cells migrating into the graft.

In one embodiment, the oxygen may be delivered via encapsulated biodegradable spheres which are associated with the tissue graft to achieve an enhanced oxygen environment within the graft. Such spheres may be delivered using a delivery device such as that described above.

In yet another embodiment of the invention, the secondary material delivery device might comprise a pressurized chamber containing secondary materials, such as in gas form. For example, the secondary material might comprise oxygen and the chamber might comprise a hyperbaric chamber. In such a configuration, a tissue graft may be located in a pressurized hyperbaric chamber for variable times, atmospheric pressures, and frequency, in which the tissue graft is exposed to secondary materials (preferably oxygen, in order to achieve an enhanced oxygen environment within the graft). The oxygenated tissue graft may be located in a sealed package, including a pressurized package, such as for delivery and later use.

In certain configurations of the invention, a tissue graft is infused with oxygen in one or more forms (gas, bubble, capsule, etc.), ex-situ, for later association with a host. In other or additional embodiments, oxygen may be provided to the tissue graft in-situ.

In some embodiments, the tissue graft may have one or more layers. As one example, secondary materials might be associated with a tissue graft and then a covering or sealing layer might be applied to one or more surfaces thereof, such as to keep the secondary material entrained in the tissue graft and/or to control the rate at which the secondary material may disassociate itself from the graft. As one example, a tissue graft may be oxygenated and then the top thereof may be sealed, thus forcing the oxygen to migrate from the graft to the sides and/or bottom, into the surrounding host tissue.

In one embodiment, a tissue graft having associated secondary materials may be associated with a host or patient in various manners. As one example, a tissue graft might be placed on top of or over an open wound. In another configuration the tissue graft might be wrapped around host tissue to increase surface contact of the host tissue or organ

In one embodiment, medicants and cellular elements may be associated with a tissue graft for the purpose of treating residual cancer cells on the margins after surgical resection.

One aspect of the invention is the use of a tissue graft having associated secondary materials to allow for transferring/transference of the secondary materials to adjacent host human tissue.

In one embodiment, the secondary materials which are associated with the tissue graft might comprise host tissue. In one configuration, the secondary materials may have the form of cellular elements from an organ donor are associated with a tissue graft (without or with an enriched oxygen environment). For example, the secondary materials might comprise transplanted thyroid tissue cells that secrete thyroxin, pancreatic cells that secrete insulin, or pituitary cells to secrete growth hormone on a long term basis.

In one embodiment, the secondary materials may comprise genetically altered DNA/RNA.

In one embodiment, the secondary material may be selected based on one or more of a healing function of phase, a function of the host tissue, an anatomical area, a disease to be treated, a surgical procedure, or other criteria.

This aspect of the invention has a number of advantages.

The present invention provides for method and apparatus allowing relevant secondary materials to be delivered to tissue graft (and when applied in-situ, to the surrounding tissue). In one configuration, the delivered secondary materials, including by type and amount, may be changed to meet physiologic needs, such as corresponding to the phase of healing, treatment, or transplantation. In one embodiment, one or more secondary materials (not limited to oxygen) may be delivered to a tissue graft ex-situ. After the tissue graft is associated with a host, additional secondary materials may be delivered to the tissue graft in-situ, including differing at different times.

The above description represents various embodiments of the present invention. However, many variations to the method and apparatus are possible without deviating from the scope of the invention. It will be understood that the above described arrangements of apparatus and the method described herein are merely illustrative of applications of the principles of this invention and many other embodiments and modifications may be made without departing from the spirit and scope of the invention as defined in the claims.

What is claimed is:

1. A method of associating secondary materials with a tissue graft, comprising the steps of:
engaging a plurality of delivery elements with an interior portion of said tissue graft;
associating a first source of at least one first secondary material with said plurality of delivery elements;
delivering, at a first time, said at least one first secondary material to said tissue graft with said plurality of delivery elements;
associating a second source of at least one second secondary material with said plurality of delivery elements, said at least one second secondary material being different than said first secondary material; and
delivering, at a second time, said at least one second secondary material to said tissue graft with said plurality of delivery elements.

2. The method in accordance with claim 1, wherein said first and second secondary materials are selected from the group consisting of: a medicant, an external cellular matrix factor, a cell, an enzyme, and an element or compound.

3. The method in accordance with claim 2, wherein the element comprises oxygen.

4. The method in accordance with claim 1, wherein said delivery elements comprise needles.

5. The method in accordance with claim 1, wherein said delivery elements comprise blades.

6. The method in accordance with claim 1, wherein said plurality of delivery elements define one or more delivery ports and said first and second secondary materials are delivered to said tissue graft through said one or more delivery ports.

7. The method in accordance with claim 1, wherein said plurality of delivery elements are associated with a base, and wherein said base is located under a wound cover.

8. The method in accordance with claim 1, wherein said plurality of delivery elements are movable between a retracted position in which said plurality of delivery elements do not engage said tissue graft and an extended position in which said plurality of delivery elements engage said tissue graft.

9. The method in accordance with claim 8, further comprising the step of moving said plurality of delivery elements to said extended position during said delivering steps and moving said plurality of delivery elements to said retracted position between said delivering steps.

10. The method in accordance with claim 1, wherein said first time and said second time comprise different times during a healing process.

11. The method in accordance with claim 1, wherein said plurality of delivery elements are associated with a mount and a cushion is associated with said mount.

12. The method in accordance with claim 1, wherein said mount has a top and a bottom, said plurality of delivery elements extend outwardly from said bottom of said mount, and said cushion extends over at least a portion of both said top and bottom of said mount.

13. The method in accordance with claim 1, further comprising the steps of modifying one or more areas of said tissue graft to create a modified tissue graft and associating said tissue graft with a host before said delivering steps.

14. The method in accordance with claim 1, wherein said plurality of delivery elements are associated with a delivery device, said delivery device associated with a wound covering.

15. The method in accordance with claim 1, further comprising the step of locating said tissue graft in a hyperbaric chamber to create an oxygenated tissue graft, and then associating said oxygenated tissue graft with a host before said delivering steps.

16. The method in accordance with claim 1, wherein said at least one first secondary material and said at least one second secondary material are selected based upon at least one of an anatomical area with which said tissue graft is associated, a disease to be treated, or a surgical procedure.

17. The method in accordance with claim 1, wherein said tissue graft comprise at least one of cadaveric human tissue, harvested human tissue, amniotic and chorionic tissue.
